(19) United States
(12) Patent Application Publication
(10) Pub. No.: US 2010/0092535 A1
(43) Pub. Date: Apr. 15, 2010

(54) Nanoporous Drug Delivery System

(75) Inventors: Brian Cook, Windsor, CA (US); James Mitchell, Windsor, CA (US); Feridun Ozdil, Santa Rosa, CA (US); Natividad Vasquez, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/249,611

(22) Filed: Oct. 10, 2008

(51) Int. Cl. A61K 9/00; A61K 39/395; A61F 2/82

(52) U.S. Cl. 424/423; 424/130.1; 623/1.42

(57) ABSTRACT

Disclosed herein are controlled release drug delivery systems. The systems comprise a medical device at least one nonoporous surface, at least one bioactive agent and optionally a biodegradable polymer. The nanoporous surfaces of the medical devices contain nanopores capable of acting as reservoirs for drugs that are controllably released.

### FIELD OF THE INVENTION

The invention disclosed herein pertains to drug delivery systems comprising medical devices which provide controlled-release of bioactive agents from nanoporous surfaces.

### BACKGROUND OF THE INVENTION

Drug releasing medical devices are desirable as a wide variety of drugs can be associated with or applied to the surface of the medical devices and subsequently released from the surface of the device after implantation of the device within the patient's body. For example, the surfaces of a catheter can be coated with antibiotics in order to prevent bacterial infection at the insertion or implantation site. Other drug-releasing medical implants include, for example, drug-releasing stents. These stents have been particularly useful because they not only provide the mechanical structure to maintain damaged blood vessel patency, but they may also release drugs into the surrounding tissue to prevent the re-narrowing of the blood vessel.

However, there remain challenges to effectively control drug delivery to the site of disease or injury via drug-releasing medical implants. Generally, bioactive agents associated with medical implants are released from the medical implants by diffusion. Alternatively, the bioactive agents can be released from the medical implants via bulk erosion. That is, those bioactive agents that are delivered to the site of implantation by a polymeric coating are released as the polymeric coating is physically or chemically eroded. Thus, given these drug-releasing mechanisms, the bioactive agents are released soon after implantation of the medical implant. While these and other methods of drug delivery have proven useful, there still remains a need for controllably releasing bioactive agents to a site of injury or disease via drug-releasing medical implants.

Nanoporous materials, materials having nanopores, through which bioactive agents can be released, can provide such controlled release medical devices.

### SUMMARY OF THE INVENTION

Described herein are drug delivery systems comprising medical devices coated at least partially with nanoporous surfaces. Methods of controlling the size of the nanopores can fine tune the drug eluting properties of the surfaces. The nanoporous surfaces can be coated with bioabsorbale polymers. In one embodiment, a vascular stent comprises drug eluting nanopores.

In one embodiment, a controlled release drug delivery system is described comprising: (a) a medical device; (b) a nanoporous surface associated with at least a portion of said medical device; and (c) at least one bioactive agent disposed within the nanopores of said nanoprous surface.

In one embodiment, the system further comprises at least one biodegradable polymer associated with said nanoporous surface. In another embodiment, the medical device is selected from the group consisting of vascular stents, esophageal stents, bile duct stents, tracheal stents, colon stents, bronchial stents, urethral stents, guide wires, pacemakers, bone screws, sutures, heart valves, and ureteral stents. In another embodiment, the medical device is a vascular stent.

In one embodiment, the nanoporous surface is selected from the group consisting of metal alloys, semiconductors, ceramics, polymers or combinations thereof. In one embodiment, the metal alloys are selected from the group consisting of nickel, cobalt, chromium, zinc, iron, ruthenium, platinum, palladium, iridium, titanium, gold, molybdenum, tungsten, tantalum, magnesium and combinations thereof.

In one embodiment, the biodegradable polymer comprises polycarbonates, polyesters, polyanhydrides, polycaprolactones, polyglycolides, polylactides, polybutyrolactones, polyethylene glycols, derivatives and combinations thereof. In another embodiment, the biocompatible polymer is a top coat. In one embodiment, the top coat comprises said at least one bioactive agent.

In one embodiment, the nanoporous surface comprises said bioactive agent. In another embodiment, the bioactive agent is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

In one embodiment, a stent is described comprising: (a) at least one nanoporous surface associated with at least a portion of said stent; and (b) at least one bioactive agent associated with said nanoporous surface. In another embodiment, the stent further comprises at least one biodegradable polymer associated with the nanoporous surface.

In another embodiment, the stent is selected from the group consisting of vascular stents, esophageal stents, bile duct stents, tracheal stents, colon stents, bronchial stents, urethral stents, and ureteral stents. In another embodiment, the stent is a vascular stent.

In one embodiment, the nanoporous surface is selected from the group consisting of metal alloys, semiconductors, ceramics, polymers or combinations thereof. In another embodiment, the metal alloys are selected from the group consisting of nickel, cobalt, chromium, zinc, iron, ruthenium, platinum, palladium, iridium, titanium, gold, molybdenum, tungsten, tantalum, magnesium and combinations thereof.

In one embodiment, the biodegradable polymer is selected from the group consisting of polycarbonates, polyesters, polyanhydrides, polycaprolactones, polyglycolides, polylactides, polybutyrolactones, polyethylene glycols, derivatives and combinations thereof. In another embodiment, the biocompatible polymer is a top coat. In another embodiment, the top coat comprises said at least one bioactive agent.

In one embodiment, the nanoporous surface comprises said bioactive agent. In another embodiment, the bioactive agent is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

### Definition of Terms

Nanoporous Materials: As used herein “nanoporous materials” consist of a regular organic or inorganic framework supporting a porous structure. The pores are in the nanometer range, between 1×10⁻⁷ meters and 0.2×10⁻⁹ meters in diameter.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

Bioactive Agent(s): As used herein, “bioactive agent” shall include any compound or drug having a therapeutic effect in an animal. Exemplary, non limiting examples include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, cytotoxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, and other rapamycin hydroxyesters as disclosed in U.S. Pat. No. 5,362,718 may be used in combination with the polymers of the present invention. All of the above references are incorporated by reference herein for all they contain regarding FKBP-12 binding agents.

### DETAILED DESCRIPTION OF THE INVENTION

The present invention pertains to nanoporous drug delivery systems comprising implantable medical devices having controlled release nanoporous surfaces capable of eluting at least one bioactive agent locally at a treatment site. The controlled-release nanoporous drug delivery systems comprise medical device substrates fabricated from nanoporous materials or medical device substrates coated with the nanoporous materials. Furthermore, the nanoporous drug delivery systems can have the nanoporous characteristic on all surfaces of the medical device, only on one surface, or a portion of a surface. For example, the nanoporous surface elutes drug only on the abluminal surface.

Nanoporous surfaces have unique physical properties. One important aspect is that a very high surface area to volume ratio can be achieved, rendering the surface capable of high amounts of drug loading. Controlling the sizes of the nanopores enables the practitioner to control the drug release rate and type of drug to be released into the physiological environment.

Nanopores include surface nanopores (i.e., nanopores that extend to the surface) or sub-surface nanopores (i.e., nanopores that do not extend to the surface, unless, for example, it does so via interconnection with surface pores). In this regard, in certain embodiments, nanopores are interconnected with each other, enhancing the ability of the nanoporous material to be used as a reservoir for the storage and delivery of bioactive agents.

In some embodiments, including various techniques discussed herein, a bioactive agent is deposited within the interconnected nanopores of a nanoporous surface concurrently with the formation of the nanoporous surface and at low temperatures. As defined herein, “low temperatures” are temperatures less than 100° C., typically less than 60° C., and in many instances room temperature (e.g., 15-35° C.). More fundamentally, the bioactive agent is deposited concurrently with the nanoporous material over time and at temperatures that do not result in degradation and loss of activity of the bioactive agent.

Nanoporous materials commonly have very high surface areas associated with them. For example, it is noted that nanoporous surfaces have significantly higher surface areas as compared to corresponding flat projected surfaces. This increase in surface area can be capitalized on in various ways. For example, in some embodiments, bioactive agents are bound or adsorbed to a nanoporous surface, thereby providing higher availability of the bioactive agent at the medical device surface than is obtained with a polished non-textured surface.

It is also noted that nanoporous regions have various characteristics that are driven by surface area. In this regard, as pore diameters reach nanometer-size dimensions, the surface area of the pores can become significant with respect to the volume of the pores. As the diameter of the pore approaches the diameter of the agent to be delivered, the surface interactions can dominate release rates. Furthermore, the amount of bioactive agent released and the duration of that release can also be affected by the depth and tortuousity of the nanopores within the nanoporous surface.

In accordance with other embodiments of the invention, nanoporous regions are created from a mixture that contains two or more metals of differing nobility and oxidizing and removing the metal(s) having lesser nobility from the mixture, thereby forming a nanoporous region. In these embodiments, the area(s) previously occupied by the metal(s) having lesser nobility are the nanoporous regions described above.

Various methods are available for oxidizing and removing the less noble metal(s) from the metal mixture, including (a) contact with an appropriate acid (e.g., nitric acid), (b) application of a voltage of sufficient magnitude and bias during immersion in a suitable electrolyte, and (c) heating in the presence of oxygen, followed by dissolution of the resultant oxide.

Examples of metals useful in the described embodiments include, but are not limited to, alloys of essentially any substantially non-oxidizing noble metal (e.g., gold, platinum, etc.) having nano-domains of essentially any metal that can be reacted and dissolved (e.g. Zn, Fe, Cu, Ag, etc.). Specific examples of suitable alloys include alloys comprising gold and silver (in which the silver is oxidized and removed), alloys comprising gold and copper (in which the copper is oxidized and removed), and so forth.

Further details concerning dealloying can be found, for example, in J. Erlebacher et al., “Evolution of nanoporosity in dealloying,” Nature, Vo. 410, 22 Mar. 2001, 450-453; A. J. Forty, “Corrosion micromorphology of noble metal alloys and depletion gilding,” Nature, Vol. 282, 6 Dec. 1979, 597-598; and R. C. Newman et al., “Alloy Corrosion,” MRS Bulletin, July 1999, 24-28.

Other aspects are directed to the formation of nanostructured regions using methods that comprise physical vapor deposition, ion deposition, ion implantation, and/or X-ray lithography. These processes are typically conducted in the presence of a substrate, which can be, for example, a metal, semiconductor, ceramic or polymer substrate.

Physical vapor deposition (PVD), ion deposition, ion implantation, and X-ray lithography are frequently carried out under vacuum (i.e., at pressures that are less than ambient atmospheric pressure). By providing a vacuum environment, the mean free path between collisions of vapor particles (including atoms, molecules, ions, etc.) is increased, and the concentration of gaseous contaminants is reduced, among other effects.

PVD processes are processes in which a source of material, typically a solid material, is vaporized, and transported to a substrate where a film (e.g., a layer) of the material is formed. PVD processes are generally used to deposit films with thicknesses in the range of a few nanometers to thousands of nanometers, although greater thicknesses are possible. PVD can take place in a wide range of gas pressures, for example, commonly within the range of 10⁻⁵ to 10⁻⁹ torr. In many embodiments, the pressure associated with PVD techniques is sufficiently low such that little or no collisions occur between the vaporized source material and ambient gas molecules while traveling to the substrate. Hence, the trajectory of the vapor is generally a straight (line-of-sight) trajectory.

Some specific PVD methods that are used to form nanostructured regions include evaporation, sublimation, sputter deposition and laser ablation deposition. For instance, in some embodiments, a source material is evaporated or sublimed, and the resultant vapor travels from the source to a substrate, resulting in a deposited layer on the substrate. Examples of sources for these processes include resistively heated sources, heated boats and heated crucibles, among others.

Sputter deposition is another PVD process, in which surface atoms or molecules are physically ejected from a surface by bombarding the surface (commonly known as a sputter target) with high-energy ions. As described supra, the resultant vapor travels from the source to the substrate where it is deposited. Ions for sputtering can be produced using a variety of techniques, including arc formation (e.g., diode sputtering), transverse magnetic fields (e.g., magnetron sputtering), and extraction from glow discharges (e.g., ion beam sputtering), among others. One commonly used sputter source is the planar magnetron, in which a plasma is magnetically confined close to the target surface and ions are accelerated from the plasma to the target surface.

In accordance some embodiments, two or more materials are co-deposited using any of several PVD processes, including evaporation, sublimation, laser ablation and sputtering. For instance, two or more materials can be co-sputtered (e.g., by sputtering separate targets of each of the materials or by sputtering a single target containing multiple materials). By co-sputtering two immiscible metals, for example, an alloy film can be formed, which is then annealed to cause phase separation and the creation of a nanostructured region having a phase domain of one metal (e.g., a matrix phase) and a separate phase domain of the other metal (e.g., a disperse phase). If desired, one metal (e.g., the nano-domains corresponding to the disperse phase) can be removed preferentially, for instance, using techniques such as those discussed above, thereby producing a nanoporous region. As another example, by co-sputtering magnetic and insulating materials, magnetic nanoparticles (e.g., Fe nanoparticles) are formed in an insulating matrix (e.g., a ceramic matrix).

In some embodiments, nucleation and growth of nanoparticles in the vapor phase prior to deposition on a substrate is achieved by sputtering at higher pressures. Moreover, in some embodiments, phase separated films from thermodynamically miscible materials are created by alternatively sputtering at low and high pressures.

Further information regarding sputtering of nanostructured films can be found in Handbook of Nanophase and Nanostructured Materials. Vol. 1. Synthesis. Wang, et al., Editors; Kluwer Academic/Plenum Publishers, Chapter 9, “Nanostructured Films and Coating by Evaporation, Sputtering, Thermal Spraying, Electro- and Electroless Deposition”.

Laser ablation deposition is another PVD process, which is similar to sputter deposition, except that vaporized material is produced by directing laser radiation (e.g., pulsed laser radiation), rather than high-energy ions, onto a source material (typically referred to as a target). The vaporized source material is subsequently deposited on the substrate.

As with other PVD processes, two materials may be co-deposited (e.g., by ablating separate targets or by ablating a single target containing a combination of materials). Moreover, in some embodiments, nucleation and growth of nanoparticles in the vapor phase prior to deposition on a substrate is achieved by ablation at higher pressures.

Because many PVD processes are low temperature processes, a thermally sensitive biologically active agent can be simultaneously co-deposited with another material (e.g., a ceramic, metallic or polymeric material), for example, using techniques such as the evaporation, sublimation, sputter deposition and laser ablation techniques described above.

In still other embodiments, nanostructured regions are produced by ion deposition processes. An “ion deposition process” is a deposition process in which ions are accelerated by an electric field, such that the substrate is bombarded with ions during the deposition process.

In some embodiments, the substrate is bombarded with ions during the course of a PVD deposition process to achieve a nanostrcutred region, in which case the technique is sometimes referred to as ion beam assisted deposition. For example, the substrate can be bombarded with ions of a reactive gas such as oxygen or nitrogen, or an inert gas such as argon, during the course of a PVD process like those discussed above. These ions can be provided, for example, by means of an ion gun or another ion beam source.

In some instances, at least a portion of the deposition vapor itself is ionized and accelerated to the substrate. For example, the deposition vapor can correspond to the material to be deposited (e.g., where a vapor produced by a PVD processes such as evaporation, sublimation, sputtering or laser ablation is ionized and accelerated to the substrate). As another example, the deposition vapor can correspond to a chemical precursor of the deposited material (e.g., where a precursor vapor for a chemical vapor deposition process such as low-pressure or plasma-enhanced chemical vapor deposition is ionized and accelerated to the substrate).

Deposition vapors can be ionized using a number of techniques. For example, deposition vapor can be at least partially ionized by passing the same through a plasma. As another example, partially ionized vapor can be directly generated at a material source, for instance, by subjecting the material source to an electronic beam and/or to an arc erosion process, such as a cathodic or an anodic arc erosion processes. Specific examples of such processes include rod cathode arc-activated deposition (RAD), spotless arc deposition (SAD), and hollow cathode activated deposition (HAD).

In other embodiments, nanostructured regions are established by subjecting an ionic species to an electric field that is sufficiently high such that the impacting ions are implanted in or beneath the substrate surface. Such “ion implantation” processes are used, for example, to create nanoclusters of a variety of materials, including metal and ceramic materials. Suitable species for ion implantation include, for example, ionic species corresponding to an element or molecule found in the substrate, ionic species corresponding to other elements or molecules not found in the substrate, including ionic species corresponding to reactive and non-reactive species (e.g., a reactive gas such as oxygen or an inert gas such as argon).

In some cases, multiple deposition techniques are combined to form nanostructured regions on medical devices. One specific example is the deposition of polymers (e.g., by plasma enhanced polymerization) concurrently with PVD-type deposition of metals to produce mixed metal-polymer films. See “Plasma Polymer-Metal Composite Films,: H. Biedermann and L. Nartinu, p. 269 in Plasma Deposition, Treatment and Etching of Polymers, Riccardo d'Agostino, Ed., Academic Press (1990). In another specific example, ion deposition is combined with ion implantation in a process known as plasma ion immersion implantation and deposition.

In still other embodiments, nanostructured regions are established via X-ray lithography. One process, known as columnated plasma lithography, is capable of producing X-rays for lithography having wavelengths on the order of 10 nm. Once a suitable mask is provided on a substrate using X-ray lithography, the substrate is subjected to a subsequent etching, deposition or reaction step, resulting in a nanostructured surface on the substrate.

Other aspects involve the use of chemical vapor deposition (CVD) to produce nanostructured regions or nanoparticles. CVD is a process whereby atoms or molecules are deposited in association with a chemical reaction (e.g., a reduction reaction, an oxidation reaction, a decomposition reaction, etc.) of vapor-phase precursor species. When the pressure is less than atmospheric pressure, the CVD process is sometimes referred to as low-pressure CVD or LPCVD. Plasma-enhanced chemical vapor deposition (PECVD) techniques are chemical vapor deposition techniques in which a plasma is employed such that the precursor gas is at least partially ionized, thereby reducing the temperature that is required for chemical reaction.

A variety of materials can be formed using CVD (including LPCVD). For example, metals can be formed using metallorganic precursors or by the reduction of metal chlorides with hydrogen. As other examples, ceramics can be formed from oxygen-containing metallic precursors, or from metallic precursors (e.g., WF₆ or TiCl₄) in the presence of oxygen or an oxygen containing species. As with CVD, a wide range of materials can be deposited with PECVD. As a specific example, monomeric precursors are frequently deposited as polymer layers using PECVD.

In some CVD processes, vapor generated from solid sources (for example, using processes like those discussed above in connection with PVD), are reacted with another species (for example, a reactive gas or another vaporized solid material) in the deposition environment. As one specific example, metal ceramics can be formed by vaporizing and depositing metal in the presence of oxygen gas at low pressure.

Several of the techniques described herein rely on the use of particles to form nanostructured regions, including nanoporous regions. Particles of numerous materials, including nanoparticles, are commercially available from a number of sources. Nanoparticles are made using various techniques, including CVD and chemical vapor condensation (CVC), which are particularly useful for the formation of metallic oxide nanoparticles.

In particle formation using CVD, gas phase nucleation and growth are controlled, typically by controlling the number of nuclei formed in the CVD reactor and by controlling the concentration of the condensing species in the gas phase. For example, supersaturation of the gas phase is frequently achieved by increasing the temperature and pressure in the reactor, while decreasing the flow rate. In particle formation using CVC, on the other hand, particles are also formed based on gas phase nucleation. In this process, metallorganic compounds are frequently used as precursor chemicals. For example, a carrier gas is bubbled through the precursor and the resulting vapor phase is introduced into a vacuum chamber, after which the metallorganic compounds pass through a heated zone. While in the heated zone the compounds begin to decompose thermally, and they begin to coalesce, thereby forming small clusters of particles. After passing though the heated zone, rapid expansion of the stream moderates particle growth and agglomeration. The particles are then condensed on a cooled surface and collected.

Other embodiments are directed to the formation of nanostructured regions, including nanoporous regions, using methods that comprise CVD. These processes are typically conducted in the presence of a substrate, which can be, for example, a metal, semiconductor, ceramic or polymer substrate. Unlike physical vapor deposition processes above, chemical vapor deposition processes are not necessarily line-of-sight processes, allowing coatings to be formed on substrates of complex geometry.

For example, in a process known as particle-precipitation-aided chemical vapor deposition (PP-CVD), an aerosol of particles is first formed by a gas phase reaction at elevated temperature. The particles are then deposited on a substrate, for example, due to the forces of electrophoresis, thermophoresis, or forced flow. In certain embodiments, a heterogeneous reaction occurs simultaneously with deposition to interconnect the particles and form a nanoporous layer, or the deposited particles are sintered to form a nanoporous layer, or both. As a specific example, a CO₂ laser can be used to heat metallorganic precursor compounds in the gas phase, resulting in decomposition of the precursor with concomitant formation of an aerosol of ceramic nanoparticles. The particles are then deposited on a substrate as a result of a thermal gradient that naturally exists between the heated reaction zone created by the laser and the cooler substrate. In this example, heterogeneous reactions at the substrate surface can be controlled independently of the gas phase reactions.

Nanoporous polymer films can also be deposited by CVD. For example, in hot-filament CVD (HFCVD, also known as pyrolytic or hot-wire CVD), a precursor gas is thermally decomposed by a resistively heated filament. The resulting pyrolysis products then adsorb onto a substrate maintained at around room temperature and react to form a film. For example, fluorocarbon films can be made using hexafluororpropylene oxide as a precursor gas. Due to the nucleation and growth mechanisms in the HFCVD processes, nanoporous films can be made using HFCVD. For further information, see, e.g., United States Patent Application No. 2003/0138645 to Gleason et al. and K. K. S. Lau et al., “Hot-wire chemical vapor deposition (HWCVD) of fluorocarbon and organosilicon thin films,” Thin Solid Films, 395 (2001) pp. 288-291.

In other embodiments, nanostructures are grown within preexisting porous layers using atomic-layer chemical vapor deposition. See, e.g., See Marian Nanu, “Nanostructured TiO₂-CuInS₂ based solar cells,” E-MRS Spring Meeting 2003, Jun. 10-13, 2003, SYMPOSIUM D, Thin Film and Nano-Structured Materials for Photovoltaics, Abstract No. D-X.2, in which CuinS₂ is applied inside the pores of nanoporous TiO₂, which comprises 10 to 50 nm particles, using atomic layer chemical vapor deposition (ALCVD). In this particular gas-phase deposition technique, reactants are supplied sequentially to avoid clogging of the nanopores.

A variety of nanostructured films can be formed by electrodeposition, including metallic, ceramic, and polymeric films. Where a metallic film is formed, the film is oxidized in certain embodiments to form a ceramic surface.

Furthermore, nanostructured regions can be formed by incorporating suspended nanoparticles into a matrix that is formed by electrodeposition. For example, nanoparticles can be dispersed by adsorbing cations on the surface of the same. During electrodeposition, the nanoparticles with adsorbed cations travel to the cathode where electrodeposition takes place, thereby incorporating the nanoparticles into the deposited layer.

Filled and unfilled nanoporous regions can be formed using such techniques. For example, in some embodiments, nanoparticles are incorporated into an electrodeposited layer which are subsequently reduced in volume or eliminated (e.g., a sublimable, evaporable, combustible or dissolvable material such as those discussed above). In other embodiments, nanoparticles of a biologically active agent are incorporated into an electrodeposited layer.

Hence, using the above and other techniques, nanostructured regions can be formed from a wide range of materials, including suitable materials selected from the metals, ceramics and polymers listed below.

Suitable materials include, but are not limited to, calcium phosphate ceramics (e.g., hydroxyapatite); calcium-phosphate glasses, sometimes referred to as glass ceramics (e.g., bioglass); metal oxides, including non-transition metal oxides (e.g., oxides of metals from groups 13, 14 and 15 of the periodic table, including, for example, aluminum oxide) and transition metal oxides (e.g., oxides of metals from groups 3, 4, 5, 6, 7, 8, 9, 10, 11 and 12 of the periodic table, including, for example, oxides of titanium, zirconium, hafnium, tantalum, molybdenum, tungsten, rhenium, iridium, and so forth); and carbon based ceramic-like materials such as silicon carbides and carbon nitrides.

Suitable metals include, but are not limited to, silver, gold, platinum, palladium, iridium, osmium, rhodium, titanium, tungsten, magnesium and ruthenium and metal alloys such as cobalt-chromium alloys, nickel-titanium alloys (e.g., nitinol), iron-chromium alloys (e.g., stainless steels, which contain at least 50% iron and at least 11.5% chromium), cobalt-chromium-iron alloys (e.g., elgiloy alloys), and nickel-chromium alloys (e.g., inconel alloys), among others.

Suitable polymer include, but are not limited to polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydoxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, polystyrenes, styrene-maleic anhydride copolymers, styrene-butadiene copolymers, styrene-ethylene-butylene copolymers (e.g., a polystyrene-polyethylene/butylene-polystyrene (SEBS) copolymer, available as Kraton.RTM. G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); glycosaminoglycans; polyesters including polyethylene terephthalates and aliphatic polyesters such as polymers and copolymers of lactide (which includes lactic acid as well as d-, I- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), poly-4-methyl-pen-1-enes, ethylene-alpha-olefin copolymers, ethylene-methyl methacrylate copolymers and ethylene-vinyl acetate copolymers; polyolefin elastomers (e.g., santoprene), ethylene propylene diene monomer (EPDM) rubbers, fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and further copolymers of the above.

Such polymers may be provided in a variety of configurations, including cyclic, linear and branched configurations. Branched configurations include star-shaped configurations (e.g., configurations in which three or more chains emanate from a single branch point), comb configurations (e.g., graft polymers having a main chain and a plurality of branching side chains), and dendritic configurations (e.g., arborescent and hyperbranched polymers). The polymers can be formed from a single monomer (e.g., they can be homopolymers), or they can be formed from multiple monomers (e.g., they can be copolymers) that can be distributed, for example, randomly, in an orderly fashion (e.g., in an alternating fashion), or in blocks.

In embodiments in which a nanostructured region is formed in or on an underlying substrate or is attached to an underlying substrate, the substrate material is typically a ceramic, metal or polymeric substrate, which can comprise suitable materials selected from those listed supra. The substrate material can also be a semiconductor (e.g., silicon). The broad range of substrate materials that can be utilized is a result of, in part, the ability to form nanostructured regions on the substrate at or near ambient temperatures or to the ability to attach previously formed nanostructured regions to the substrate.

According to various aspects, biologically active agents are disposed on and/or within a range of nanostructured regions, including nanoporous regions and nanotextured regions.

As noted above, biologically active agents are loaded for any of a number of purposes, for example, to effect in vivo release of the biologically active agents (which may be, for example, immediate or sustained release), to influence (e.g., either promote or inhibit) bonding between the medical device and adjacent tissue, to influence thromboresistance, to influence antihyperplastic behavior, to enhance recellularization, and to promote tissue neogenesis, among many other purposes.

The medical devices can be loaded with biologically active agents such the biologically active agents are released, retained or both upon contact with a patient.

For example, in embodiments where tortuous paths are created by an interconnected nanoporous network and/or where pore diameters approach the size of the agent to be delivered, release of biologically active agents can be significantly delayed, in some instances approaching zero order release kinetics.

As another example, in embodiments where surface features associated with nanostructured regions are filled with biologically active agents that are retained upon patient contact, nano-sized areas of the biologically active agents are created in some instances to control cellular interactions and adhesion.

As noted above, nanostructured regions (including nanoporous regions and nanotextured surface regions), whether with or without biologically active agents, can correspond to the entire medical device surface, or to only a portion (or portions) of the medical device. Hence, one or more nanostructured regions can be provided on the medical device surface at desired locations and/or in desired shapes (e.g., in desired patterns, for instance, using appropriate masking techniques, including lithographic techniques). For example, for tubular devices such as stents (which can comprise, for example, a laser or mechanically cut tube, one or more braided, woven, or knitted filaments, etc), the nanostructured regions can be provided on the luminal surfaces, on the abluminal surfaces, on the lateral surfaces between the luminal and abluminal surfaces, patterned along the luminal or abluminal length of the devices, on the ends, and so forth. Moreover, multiple nanostructured regions can be formed using the same or different techniques, and can contain the same biologically active agent, different biologically active agents, or no biologically active agent. It is therefore possible, for example, to release the same or different therapeutic agents at different rates from different locations on the medical device. As another example, it is possible to provide a tubular medical device (e.g., a vascular stent) having a first nanoporous region comprising a first biologically active agent (e.g., an antithrombotic agent) on its inner, luminal surface and a second nanoporous region comprising a second biologically active agent that differs from the first biologically active agent (e.g., an antiproliferative agent) on its outer, abluminal surface (as well as on the ends).

Many size altering methods can be used to direct the dimensions of the nanopores including, but not limited to plasma etching, chemical etching, irradiation with electromagnetic radiation, chemical vapor deposition (CVD), and precise manufacturing processes. Furthermore, the nanoporous surface can comprise a nanoporous coating applied to a medical device.

One embodiment is directed to implantable medical devices having therapeutic agents associated therewith. These medical devices include, but are not limited to, stents, catheters, micro-particles, probes, vascular grafts, access devices, in-dwelling access ports, valves, plates, barriers, supports, shunts, discs, joints, as well as virtually any device intended for temporary or permanent implantation including implants that are bioresorbed. In one embodiment, the medical device is a vascular stent.

In one embodiment, the diameters of the nanopores are less than 100 nm, preferably, less than 75 nm, more preferably less than 50 nm. In other embodiments, the diameters of the nanopores range from about 10 nm to about 200 nm. In one embodiment the diameters range from about 15 nm to about 190 nm. In one embodiment the diameters range from about 20 nm to about 180 nm. In one embodiment the diameters range from about 25 nm to about 170 nm. In one embodiment the diameters range from about 30 nm to about 160 nm. In one embodiment the diameters range from about 35 nm to about 150 nm. In one embodiment the diameters range from about 40 nm to about 140 nm. In one embodiment the diameters range from about 45 nm to about 130 nm. In one embodiment the diameters range from about 50 nm to about 120 nm. In one embodiment the diameters range from about 55 nm to about 110 nm. In one embodiment the diameters range from about 60 nm to about 100 nm. In one embodiment the diameters range from about 65 nm to about 90 nm. In one embodiment the diameters range from about 70 nm to about 85 nm. In one embodiment the diameters range from about 75 nm to about 80 nm.

The depth of the nanopores can also be controlled through standard methods known to those ordinarily skilled in the art. Increasing the depth of the nanopores, thereby increasing the volume, enables the practitioner to load higher amounts of drugs. Furthermore, a relatively deep nanopore can be loaded with a minor amount of drug allowing a slower release of the drug into the physiological atmosphere.

The controlled release medical devices described herein can be coated with biocompatible biodegradable polymers. Once the nanopores are loaded with the appropriate bioactive agent a biodegradable polymeric controlled release top coat can be optionally applied over the nanoporous surface. In one embodiment a bioactive agent is controllably released into the physiological atmosphere while the polymeric top coat is biodegraded exposing the nanoporous surface. This above strategy allows for a plurality of bioactive agents to be eluted at different times.

In one embodiment, the bioactive agent eluting medical device is coated with a polymeric topcoat. The polymeric top coats include, but are not limited to, polycarbonates, polyesters, polyanhydrides, polycaprolactones, polyglycolides, polylactides, polybutyrolactones, polyethylene glycols, and derivatives and copolymers thereof. The top coat polymers may optionally contain bioactive agents that are the same or different from the bioactive agents present on the nanoporous medical device surface being coated. This strategy allows for a plurality of bioactive agents being eluted from the implanted medical device, each bioactive agent being eluted at different times.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A controlled release drug delivery system comprising:
(a) a medical device;
(b) a nanoporous surface associated with at least a portion of said medical device; and
(c) at least one bioactive agent disposed within the nanopores of said nanoprous surface.

2. The controlled release drug delivery system according to claim 1 further comprising at least one biodegradable polymer associated with said nanoporous surface.

3. The controlled release drug delivery system according to claim 1 wherein said medical device is selected from the group consisting of vascular stents, esophageal stents, bile duct stents, tracheal stents, colon stents, bronchial stents, urethral stents, guide wires, pacemakers, bone screws, sutures, heart valves, and ureteral stents.

4. The controlled release drug delivery system according to claim 1 wherein said medical device is a vascular stent.

5. The controlled release drug delivery system according to claim 1 wherein said nanoporous surface is selected from the group consisting of metal alloys, semiconductors, ceramics, polymers or combinations thereof.

6. The controlled release drug delivery system according to claim 5 wherein said metal alloys are selected from the group consisting of nickel, cobalt, chromium, zinc, iron, ruthenium, platinum, palladium, iridium, titanium, gold, molybdenum, tungsten, tantalum, magnesium and combinations thereof.

7. The controlled release drug delivery system according to claim 1 wherein said biodegradable polymer comprises polycarbonates, polyesters, polyanhydrides, polycaprolactones, polyglycolides, polylactides, polybutyrolactones, polyethylene glycols, derivatives and combinations thereof.

8. The controlled release drug delivery system according to claim 1 wherein said biocompatible polymer is a top coat.

9. The controlled release drug delivery system according to claim 8 wherein said top coat comprises said at least one bioactive agent.

10. The controlled release drug delivery system according to claim 1 wherein said nanoporous surface comprises said bioactive agent.

11. The controlled release drug delivery system according to claims 9 or 10 wherein said bioactive agent is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

12. A stent comprising:
(a) at least one nanoporous surface associated with at least a portion of said stent; and
(b) at least one bioactive agent associated with said nanoporous surface.

13. The stent according to claim 12 further comprising at least one biodegradable polymer associated with said nanoporous surface.

14. The stent according to claim 12 wherein said stent is selected from the group consisting of vascular stents, esophageal stents, bile duct stents, tracheal stents, colon stents, bronchial stents, urethral stents, and ureteral stents.

15. The stent according to claim 12 wherein said stent is a vascular stent.

16. The stent according to claim 12 wherein said nanoporous surface is selected from the group consisting of metal alloys, semiconductors, ceramics, polymers or combinations thereof.

17. The stent according to claim 16 wherein said metal alloys are selected from the group consisting of nickel, cobalt, chromium, zinc, iron, ruthenium, platinum, palladium, iridium, titanium, gold, molybdenum, tungsten, tantalum, magnesium and combinations thereof.

18. The stent according to claim 12 wherein said biodegradable polymer is selected from the group consisting of polycarbonates, polyesters, polyanhydrides, polycaprolactones, polyglycolides, polylactides, polybutyrolactones, polyethylene glycols, derivatives and combinations thereof.

19. The stent according to claim 1 wherein said biocompatible polymer is a top coat.

20. The stent according to claim 19 wherein said top coat comprises said at least one bioactive agent.

21. The stent according to claim 12 wherein said nanoporous surface comprises said bioactive agent.

22. The controlled release drug delivery system according to claims 20 or 21 wherein said bioactive agent is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.
